(19) United States
(12) Patent Application Publication
(10) Pub. No.: US 2025/0391165 A1
(43) Pub. Date: Dec. 25, 2025

(54) IMAGE PROCESSING APPARATUS, IMAGE PROCESSING METHOD, AND STORAGE MEDIUM

(71) Applicant: OLYMPUS MEDICAL SYSTEMS CORP., Tokyo (JP)

(72) Inventors: Takeshi OTSUKA, Tokyo (JP); Akihiro KUBOTA, Tokyo (JP)

(73) Assignee: OLYMPUS MEDICAL SYSTEMS CORP., Tokyo (JP)

(21) Appl. No.: 19/316,063

(22) Filed: Sep. 2, 2025

Related U.S. Application Data
(63) Continuation of application No. PCT/JP2023/007884, filed on Mar. 2, 2023.

(51) Int. Cl. G06V 10/70; G06T 7/00; G06V 10/82; G16H 50/30

(52) U.S. Cl. CPC G06V 10/87; G06T 7/0016; G06V 10/82; G16H 50/30; G06T 2207/10068; G06T 2207/30096

(57) ABSTRACT

An image processing apparatus includes one or more processors. The one or more processors store one or more first machine learning models in a setting space including a first setting condition and a second setting condition, the one or more first machine learning models being placed at or below a predetermined density in the setting space, receives an image for identification, selects one or more second machine learning models that, in the setting space, are either within a predetermined range from the image for identification, or in order of shorter distance from the image for identification, and identifies the image for identification using the second machine learning models.

### CROSS REFERENCE TO RELATED APPLICATION

This application is a continuation application of PCT/JP2023/007884 filed on Mar. 2, 2023, the entire contents of which are incorporated herein by this reference.

### BACKGROUND OF THE INVENTION

### 1. Field of the Invention

The present disclosure relates to an image processing apparatus for performing image identification processing using a machine learning model, an image processing method for performing the image identification processing using the machine learning model, and a program for causing the image processing apparatus to perform the image identification processing using the machine learning model.

### 2. Description of the Related Art

Development is underway for image processing methods that use an image processing apparatus to identify a lesion included in an inputted image, such as an endoscopic image photographed by an endoscope or a pathology image of a pathology specimen photographed by a microscope, by AI using a machine-learned neural network model (hereinafter referred to as a “model”). In the identification technique using AI, e.g., endoscopic image identification processing, an image for identification is created in various parameter sets.

An image parameter set is a combination of setting conditions, such as a photographing parameter and an image processing parameter. Therefore, to ensure high identification accuracy in AI identification processing of images, it is preferred to create a dedicated model using dedicated training data for each parameter set, which is a combination of a plurality of parameters.

Japanese Patent Publication No. 2021-149640 discloses a data classification method including synthesizing data outputted from a plurality of models.

### SUMMARY OF THE INVENTION

An image processing apparatus according to an embodiment of the present disclosure includes one or more processors. The one or more processors store, in a setting space including a first setting condition and a second setting condition, at least one of: one or more first machine learning models, the number of which is less than a total number of combinations of the first setting condition and the second setting condition; or a plurality of first thresholds, the number of which is less than the total number of combinations of the first setting condition and the second setting condition, the at least one of the one or more first machine learning models or the plurality of first thresholds being placed at or below a predetermined density in the setting space, receives an image for identification inputted as an identification target, selects, from among the at least one of the one or more first machine learning models or the plurality of first thresholds, at least one of: one or more second machine learning models that, in the setting space, are either within a predetermined range from the image for identification, or in order of shorter distance from the image for identification; or a plurality of second thresholds that, in the setting space, are either within the predetermined range from the image for identification, or in order of shorter distance from the image for identification, and identifies the image for identification using the at least one of the one or more second machine learning models or the plurality of second thresholds.

An image processing method according to an embodiment of the present disclosure includes one or more processors storing, in a setting space including a first setting condition and a second setting condition, at least one of: one or more first machine learning models, the number of which is less than a total number of combinations of the first setting condition and the second setting condition; or a plurality of first thresholds, the number of which is less than the total number of combinations of the first setting condition and the second setting condition, the at least one of the one or more first machine learning models or the plurality of first thresholds being placed at or below a predetermined density in the setting space; the one or more processors receiving an image for identification inputted as an identification target; the one or more processors selecting, from among the at least one of the one or more first machine learning models or the plurality of first thresholds, at least one of: one or more second machine learning models that, in the setting space, are either within a predetermined range from the image for identification, or in order of shorter distance from the image for identification; or a plurality of second thresholds that, in the setting space, are either within the predetermined range from the image for identification, or in order of shorter distance from the image for identification; and the one or more processors identifying the image for identification using the at least one of the one or more second machine learning models or the plurality of second thresholds.

A non-transitory computer-readable storage medium according to an embodiment of the present disclosure stores a program for an image processing apparatus. One or more processors of the image processing apparatus stores, in a setting space including a first setting condition and a second setting condition, at least one of: one or more first machine learning models, the number of which is less than a total number of combinations of the first setting condition and the second setting condition; or a plurality of first thresholds, the number of which is less than the total number of combinations of the first setting condition and the second setting condition, the at least one of the one or more first machine learning models or the plurality of first thresholds being placed at or below a predetermined density in the setting space, and the program is configured such that: the one or more processors receives an image for identification inputted as an identification target; the one or more processors selects, from among the at least one of the one or more first machine learning models or the plurality of first thresholds, at least one of: one or more second machine learning models that, in the setting space, are either within a predetermined range from the image for identification, or in order of shorter distance from the image for identification; or a plurality of second thresholds that, in the setting space, are either within the predetermined range from the image for identification, or in order of shorter distance from the image for identification; and the one or more processors identifies the image for identification using the at least one of the one or more second machine learning models or the plurality of second thresholds.

### BRIEF DESCRIPTION OF THE DRAWINGS

FIG. 1 is a configuration diagram of an endoscope system including an image processing apparatus according to a first embodiment.

FIG. 2 is a flowchart of model creation processing of the image processing apparatus according to the first embodiment.

FIG. 3 is a flowchart of identification processing of the image processing apparatus according to the first embodiment.

FIG. 4 is a placement diagram of an image parameter setting space of the image processing apparatus according to the first embodiment.

FIG. 5 is a placement diagram of the image parameter setting space of the image processing apparatus according to the first embodiment.

FIG. 6 is an endoscopic image of the image processing apparatus according to the first embodiment.

FIG. 7 is an endoscopic image of the image processing apparatus according to the first embodiment.

FIG. 8A is a placement diagram of the image parameter setting space of the image processing apparatus according to the first embodiment.

FIG. 8B is a placement diagram of the image parameter setting space of the image processing apparatus according to the first embodiment.

FIG. 8C is a placement diagram of the image parameter setting space of the image processing apparatus according to the first embodiment.

FIG. 8D is a placement diagram of the image parameter setting space of the image processing apparatus according to the first embodiment.

FIG. 9 is a flowchart of identification processing of an image processing apparatus according to a second embodiment.

FIG. 10 is a flowchart of identification processing of an image processing apparatus according to a third embodiment.

FIG. 11 is a placement diagram of an image parameter setting space of the image processing apparatus according to the third embodiment.

FIG. 12 is a placement diagram of an image parameter setting space of an image processing apparatus according to a third modification example.

FIG. 13 is a placement diagram of the image parameter setting space of the image processing apparatus according to the third modification example.

### DETAILED DESCRIPTION OF THE PREFERRED EMBODIMENTS

### First Embodiment

Hereinafter, an image processing apparatus 1 according to the present embodiment will be described with reference to the drawings.

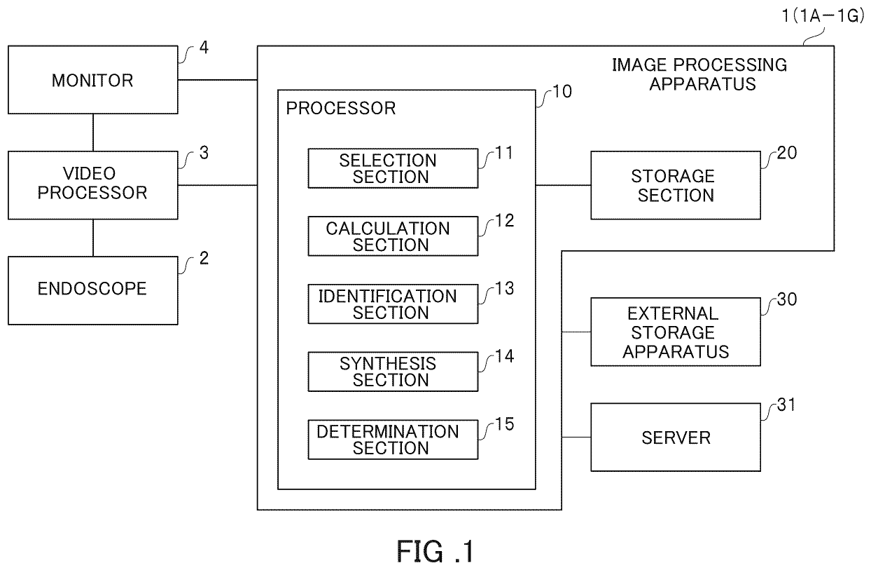

As shown in FIG. 1, an image photographed by an endoscope 2 is processed by a video processor (endoscope processor) 3 and displayed on a monitor 4. While operating the endoscope 2, a user detects a lesion region based on an endoscopic image displayed on the monitor 4. The image processing apparatus 1 is an AI system that includes a machine-learned model to assist the user to detect the lesion region.

The image processing apparatus 1 is a computer including a processor 10 and a storage section (memory) 20. The image processing apparatus 1 may include one or more processors. The processor 10 including a CPU configures a plurality of function sections that each execute a predetermined function by reading a program and a machine-learned model (hereinafter also referred to as a “model”) that are stored in the storage section 20. That is, the processor 10 includes a selection section 11, a calculation section 12, an identification section 13, a synthesis section 14, and a determination section 15. The processor 10 may include the storage section 20.

At least one of the plurality of function sections of the processor 10 may be configured by a dedicated hardware circuit. The program and the model may be stored in a server 31 via a network. The image processing apparatus 1 and the video processor 3 may be connected via the network.

### Machine Learning Model Creation

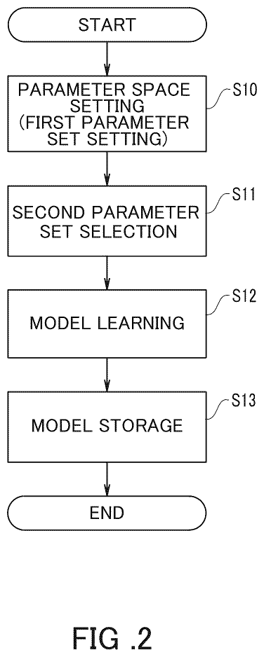

In the image processing apparatus 1, before identification processing starts, a plurality of learned models are created in advance, and the learned models are stored in the storage section 20, for example. A creation method of the learned models is described below along the flowchart in FIG. 2.

### <Step S10> Parameter Setting Space Setting

The endoscopic image is obtained under a combination of a plurality of image parameters (setting conditions) (hereafter referred to as a “parameter set”).

Each parameter (setting condition) is classified into a predetermined number of parameter levels in a setting space. If the parameter does not match a set parameter level, the parameter is processed by rounding off, for example. For example, a parameter with a level of color tone R of “1.3” is processed as parameter level “1” in the setting space.

The following are examples of parameters.
Model: 100 series, 200 series, 1500 series
Observation light: WLI (White Light Imaging), NBI (Narrow Band Imaging), and RDI (Red Dichromatic Imaging)
Structure enhancement processing: processing A (A1 to A8) and processing B (B1 to B8)
TXI (Texture and Color Enhancement Imaging): TXI1 (HIGH to LOW) and TXI2 (HIGH to LOW)
Color tone: R (R−8 to R8), B (B−8 to B8), and saturation (−8 to +8)

Color transformation parameters (bias level, contrast level, gamma level), geometric transformation parameters (sharpening processing level, blur correction level, distortion correction level), and the like may also be set as image parameters.

Note that object data and detection target data may be set as image parameters.
Objects: upper gastrointestinal tract (pharynx, esophagus, stomach, and duodenum), and lower gastrointestinal tract (small intestine, large intestine, and rectum)
Detection targets: cancer (adenocarcinoma, squamous cell carcinoma, and small cell carcinoma), inflammation, blood vessels, bleeding points, nerves, fat, ureter, or urethra

From the plurality of image parameters described above, K image parameters are selected for the identification processing. There is no particular restriction on the number K of the image parameters to be selected. However, by prioritizing industrially important image parameters, the number K of the image parameters to be selected may preferably be greater than or equal to two, and may more preferably be greater than or equal to three, for example. The number K of the image parameters may preferably be less than or equal to five.

When K image parameters are selected, a K-dimensional parameter setting space having K parameter axes is set.

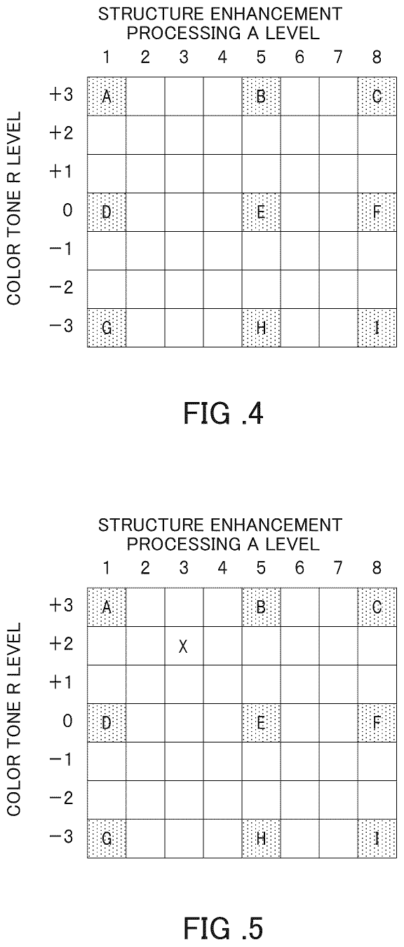

For the sake of simplicity, description will be made using an example of a two-dimensional setting space (setting plane) having two parameter axes, shown in FIG. 4. Processing A of structure enhancement processing, which is a first setting condition, has eight levels of (A1 to A8). Color tone R, which is a second setting condition, has seven levels of (−3 to 3). Therefore, the maximum number (total number) N of parameter sets (first parameter sets) that can be placed in this setting space, each being a combination of the first setting condition and the second setting condition, is 56.

In the present embodiment, although the first setting condition has eight levels and the second setting condition has seven levels, the number of levels can be set as appropriate for each parameter. The upper and lower limits of each parameter value are the upper and lower limits of each parameter value of the specifications of the endoscope or the endoscope processor.

Hereafter, for example, a parameter set in which the processing A is at level 5 and the color tone R is at level 0 is referred to as a parameter set (A5:R0).

### <Step S11> Second Parameter Set Selection

From among a plurality of first parameter sets placed in the setting space, a plurality of second parameter sets (vicinity parameter sets) based on which a model is created are selected in the selection section 11.

In FIG. 4, nine (M=0.16N) second parameter sets A to I are placed in a space where a total of fifty-six first parameter sets (N=56) can be placed. The second parameter sets may be selected automatically by the selection section 11 based on a predetermined condition or by a user.

The plurality of second parameter sets are selected so as to have a placement density in the setting space that is less than or equal to a predetermined density.

The upper limit of the number M of the second parameter sets may preferably be less than or equal to 50% of the number N of the first parameter sets, and may particularly preferably be less than or equal to 30% of the number N of the first parameter sets. The smaller number of the second parameter sets allows for reducing the amount of work that is required to prepare models corresponding to the second parameter sets. If the number M of the second parameter sets is less than or equal to the upper limit described above, the image processing apparatus 1 is highly efficient and has a small hardware load. The lower limit of the number M of the second parameter sets may preferably be greater than or equal to 5% of the number N of the first parameter sets, and may particularly preferably be greater than or equal to 10% of the number N of the first parameter sets. If the number M of the second parameter sets is greater than or equal to the lower limit described above, the image processing apparatus 1 provides high identification reliability.

The plurality of second parameter sets are placed in a dispersed state in the setting space. For example, a virtual frame (also referred to as a “kernel”) having a size of 30% of that of the setting space is set. No matter where this frame is positioned in the setting space, the number M of the second parameter sets is less than or equal to 40%, and may preferably be less than or equal to 20%, of the number N of first parameter sets in the frame. However, a cohesive area, where the second parameter sets are partially adjacent to each other or where the plurality of second parameter sets are placed at positions closer together than in surrounding areas, may be formed in the setting space.

Note that the shape of the frame is not particularly limited. When the setting space is two-dimensional, the shape of the frame is, for example, a circle, an ellipse, a rectangle, a square, or a polygon. When the setting space is three-dimensional, the shape of the frame is, for example, a sphere, a hemisphere, a cube, or a cone.

The plurality of second parameter sets placed in a dispersed manner in the setting space may preferably have uniform intervals in the setting space of at least one of the parameter levels.

For example, on a color tone R axis, the color tone R level of the second parameter set A is (R+3), the color tone R level of the second parameter set D is (R0), and the color tone R level of the second parameter set G is (R−3). The interval between the level of the second parameter set A and the level of the second parameter set D, and the interval between the level of the second parameter set D and the level of the second parameter set G, is the same: two levels.

The procedure for selecting a plurality of second parameter sets may preferably be to first select a representative parameter set and then select a plurality of parameter sets at a predetermined distance from the representative parameter set. If more second parameter sets are to be selected, a plurality of parameter sets at a predetermined distance from the selected parameter sets are selected.

The representative parameter set may be, for example, a parameter set that is obtained and preset during the development phase of the product, or may be a parameter set that is expected to be used most frequently in the user's environment. The frequency of use may be aggregated based on the parameter sets applied to a plurality of images collected as training data.

The second parameter sets are placed in the setting space in a substantially uniformly dispersed manner. Therefore, as described below, the reliability of the identification processing is ensured no matter where a third parameter set of the image for identification is positioned in the setting space.

For example, the second parameter sets may be placed uniformly or substantially uniformly throughout the setting space. In this case, there is an advantage that it is possible to produce an output with a consistent accuracy for any given identification image.

### <Step S12> Model Learning

M learned models (first machine learning models) are created that correspond respectively to M second parameter sets selected. That is, the M learned models are created by learning, for example, deep learning, using a plurality of images (training data).

The correct value in the training data stores a positive probability (also referred to as an “identification result” in the case of a value outputted by the AI model) for identifying a positive area (lesion region) or a negative area (normal region) included in the image.

For example, model learning is performed by preparing, as training data, a large number of images that are decided to be normal (positive probability P=0) and a large number of images in which a cancer is photographed as a lesion region (positive probability P=1), and inputting the prepared images into the neural network. As the training data, data-augmented (data-expanded) images may be used.

An optimal model for a certain condition may be created by learning using, as the training data, only images obtained under a certain condition and data-augmented images of the obtained images. Alternatively, without limiting to a certain condition, the learning may be performed by using, as the raining data, only images obtained under a plurality of conditions close to the certain condition and data-augmented images of the obtained images. That is, by learning only images under an arbitrary setting condition, the optimal model for the arbitrary setting condition can be created.

### <Step S13> Model Storage

The created M first machine learning models are stored in the storage section 20, for example.

That is, the image processing apparatus 1 stores, in the setting space including the first setting condition and the second setting condition, a plurality of first machine learning models, the number of which is less than the total number of combinations of the first setting condition and the second setting condition. The first plurality of machine learning models are placed at or below a predetermined density in the setting space.

As long as the processor 10 of the image processing apparatus 1 is in a state where a predetermined model can be used, the storage section 20 may be a storage section of the processor 10, of an external storage apparatus 30, or of the server 31. Furthermore, the model learning may be performed using a computer different from the image processing apparatus 1.

A model selected by the user from among models created by a third party and stored in the external storage apparatus 30, the server 31, or the like may be transferred to the processor 10 and used in the identification processing. For example, if there are already a plurality of learned models, and the parameters of the plurality of learned models can be placed at or below a predetermined density in the setting space, those created learning models may be used.

### Identification Processing

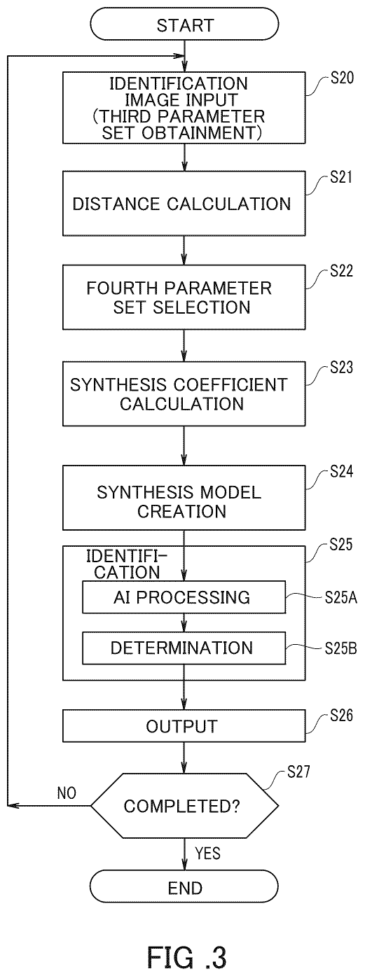

Identification processing in the image processing apparatus 1 will be described along the flowchart in FIG. 3.

### <Step S20> Image for Identification Input

An image of an object photographed by the endoscope 2 is processed by the video processor 3 and displayed on the monitor 4 as an endoscopic image. The processor 10 of the image processing apparatus 1 also receives the endoscopic image. The image received by the processor 10 may be the same as or different from the image displayed on the monitor 4. For example, a white light image may be displayed on the monitor 4 and a special light image may be inputted to the processor 10.

The processor 10 may obtain image pickup conditions from the endoscope 2 or the video processor 3. The image pickup conditions may be manually inputted into the processor 10 by a person. These image pickup conditions correspond to the parameters described above.

For example, thirty endoscopic images are outputted to the monitor 4 per second, and displayed on the monitor 4 as a moving image. The transfer rate of the endoscopic images inputted to the processor 10 may be, for example, one image per second which is less than the transfer rate at which the endoscopic images are inputted to the monitor 4, depending on the processing power of the processor 10.

From the endoscopic image (image data, the image for identification, and a third image), which is an identification target inputted from the video processor 3 to the image processing apparatus 1, data of the parameter set (the third parameter set and a parameter set for identification) of that image may be analyzed. When the image pickup conditions are obtained as described above, the parameters may be identified from among the image pickup conditions and tied to the endoscopic image. For example, in the example shown in FIG. 5, the third parameter set of the inputted endoscopic image is X (A3, B+2) in the setting space.

The image processing apparatus 1 calculates, based on the parameter information (third parameter set) possessed by the video processor 3, distances to a plurality of machine learning models (fourth parameter sets) in the setting space.

Some of the conditions of the third parameter set may be inputted by the user or may be calculated by the image processing apparatus 1 from the endoscopic image.

### <Step S21> Distance Calculation

Respective distances L are calculated between the third parameter set X and a plurality of second parameter sets A to I in the setting space. For example, the Manhattan distance L(X−A) between the third parameter set X and the second parameter set A is calculated to be “three”. In a similar manner, L(X−B), L(X−C), L(X−D), L(X−E), L(X−F), L(X−G), L(X−H), and L(X−I) are calculated.

The distance L in space may be any of the Euclidean distance, Mahalanobis distance, Manhattan distance, and Chebyshev distance.

For example, the Euclidean distance L between a parameter set A (w, x, y, z) and a parameter set B (w+Δw, x+Δx, y+Δy, z+Δz) in a four-dimensional space is calculated by L2=Δw2+Δx2+Δy2+Δz2. When used as the distance L, the Mahalanobis distance is calculated using the coordinates of the parameter set to be compared with, instead of using the mean vector.

The method for calculating the distance L may vary depending on the dimension of the setting space. For example, a multidimensional setting space may be considered as a set of a plurality of lower dimensional setting spaces, and the average of the distances calculated in respective lower dimensional setting spaces may be used as the distance L.

### <Step S22> Fourth Parameter Set Selection

In the setting space, K fourth parameter sets are selected from among the second parameter sets in order of shorter distance L from the third parameter set. K is an integer greater than or equal to one.

For example, in the setting space shown in FIG. 5, four second parameter sets A, B, D, and E are selected as the fourth parameter sets, in order of shorter distance L. An image corresponding to the fourth parameter set is referred to as a second image. In other words, a plurality of second images are selected from among a plurality of first images corresponding to the second parameter sets.

When model synthesis is performed, the number K of the fourth parameter sets may preferably be greater than or equal to two, and may particularly preferably be greater than or equal to three. The number K of the fourth parameter set may preferably be less than or equal to twenty, and may more preferably be less than or equal to ten. If the number K of the fourth parameter sets is greater than or equal to the range described above, the image processing apparatus 1 provides high identification reliability. If the number K of the fourth parameter sets is less than or equal to the range described above, the image processing apparatus 1 is highly efficient and has a low hardware load.

Note that if there are plurality of second parameter sets with the same distance L, only one of the second parameter sets may be selected as the fourth parameter set, or the fourth parameter sets may be selected beyond a predetermined number K.

For example, when the third parameter set of the endoscopic image inputted in the setting space shown in FIG. 5 is (A7, B+2), in the image processing apparatus 1 in which two fourth parameter sets are set to be selected, two second parameter sets B and C, two second parameter sets C and F, or three second parameter sets B, C, and F are selected.

Note that it goes without saying that if any of the second parameter sets exist at the same position as the third parameter set in the setting space, only the second parameter set at the same position as the third parameter set is selected as the fourth parameter set. In this case, the following synthesis coefficient need not be calculated.

### <Step S23> Synthesis Coefficient Calculation

The calculation section 12 calculates synthesis coefficients k (model synthesis coefficients), each of which is a weighting coefficient corresponding to the distance L, for each of the fourth parameter sets. Specifically, each synthesis coefficient k is calculated so that a weight w of the fourth parameter set with a shorter distance Lis larger. For example, the weighting in the synthesis coefficient k is inversely proportional to the distance L.

For example, the synthesis coefficients kA to kE of models (second machine learning models) A, B, D, and E corresponding to a fourth parameter set are calculated using the Manhattan distance L as follows.

### Manhattan Distance L

$$L ( X - A ) = 3 , L ( X - B ) = 3 , L ( X - D ) = 4 , L ( X - E ) = 4$$

### Weight w

$$wA = 1 / 3 , wB = 1 / 3 , wD = 1 / 4 , wE = 1 / 4$$

### Weighted Sum wALL

$$wALL = wA + wB + wD + wE = ( 1 / 3 ) + ( 1 / 3 ) + ( 1 / 4 ) + ( 1 / 4 ) = 14 / 12$$

### Synthesis Coefficient k

$$kA = wA / wALL = ( 1 / 3 ) / ( 14 / 12 ) = 4 / 14$$

$$kB = wB / wALL = ( 1 / 3 ) / ( 14 / 12 ) = 4 / 14$$

$$kD = wD / wALL = ( 1 / 4 ) / ( 14 / 12 ) = 3 / 14$$

$$kE = wE / wALL = ( 1 / 4 ) / ( 14 / 12 ) = 3 / 14$$

The weight w in the synthesis coefficient k may be logarithmically or exponentially inversely proportional to the distance L.

In the example described above, the weight w is calculated by applying one distance L to one parameter set. In contrast, if the multidimensional setting space is regarded as a set of a plurality of low-dimensional setting spaces, and a plurality of distances or weights calculated in the respective low-dimensional setting spaces are calculated, a weight may be calculated from each of the plurality of distances, and a value obtained by multiplying those weights together may be used as the weight w.

### <Step S24> Model Synthesis

The synthesis section 14 of the image processing apparatus 1 is a model synthesis section that synthesizes a plurality of models (the second machine learning models) A, B, D, and E corresponding to the fourth parameter sets A, B, D, and E, according to the synthesis coefficient k for each weighting. Being a weighted average by the synthesis coefficients k, the model synthesis is low-load processing that can be processed in a short time.

Although the model includes a plurality of layers, the synthesis coefficients may be different for each layer. For example, the synthesis section 14 may weight and synthesize only an output layer and an intermediate layer on the output layer side of each of the plurality of models according to the synthesis coefficients k. For example, if a model corresponding to the fourth parameter set E is the representative model, an input layer and an intermediate layer on the input layer side may use the values of the representative model as they are, and the output layer and the intermediate layer on the output layer side may be weighted and synthesized according to the synthesis coefficients k. According to the method described above, in the input layer and the intermediate layer on the input layer side using the representative model, common feature values can be extracted from the inputted images, and in the output layer and the intermediate layer on the output layer side using a synthesis model, an output can be generated from the extracted common feature values in consideration of the effects according to the parameter set. Furthermore, since common values are used on the input layer side, the model can be developed at low cost.

The representative model may be synthesized with the synthesis model. In doing so, the synthesis may be performed with a different synthesis coefficient for each layer. For example, in the input layer, the synthesis may be performed by setting the synthesis coefficient of the representative model to 0.9, and the synthesis coefficient of the synthesis model to 0.1. In the intermediate layer, the synthesis may be performed by decreasing the synthesis coefficient of the representative model and increasing the synthesis coefficient of the synthesis model as the transition is made from the input layer side to the output layer side. In the output layer, the synthesis may be performed by setting the synthesis coefficient of the representative model to 0.1, and the synthesis coefficient of the synthesis model to 0.9. In the method described above, the representative model and the synthesis model may be smoothly used together.

### <Step S25> Identification Processing

The identification section 13 performs identification processing on the inputted image for identification (the third image) using the synthesis model. The likelihood that the image for identification includes a lesion region is outputted as a positive probability P(0≤P≤1) (S25A). The positive probability P=0 means the likelihood is 0%, and the positive probability P=1 means the likelihood is 100%. If the positive probability P is greater than or equal to a predetermined threshold T for determination, the determination section 15 determines that the image for identification includes the lesion region (S25B). For example, if the threshold Tis 0.5, which is the standard threshold that is normally used, and the positive probability P is 0.8, it is determined that the image for identification includes the lesion region.

The identification is determined for each pixel of the image, for each area that includes a plurality of pixels, or by treating the entire image including the lesion region as one area.

### <Step S26> Output

The processor 10 outputs a determination result, which is an estimated value estimated based on the positive probability and the threshold.

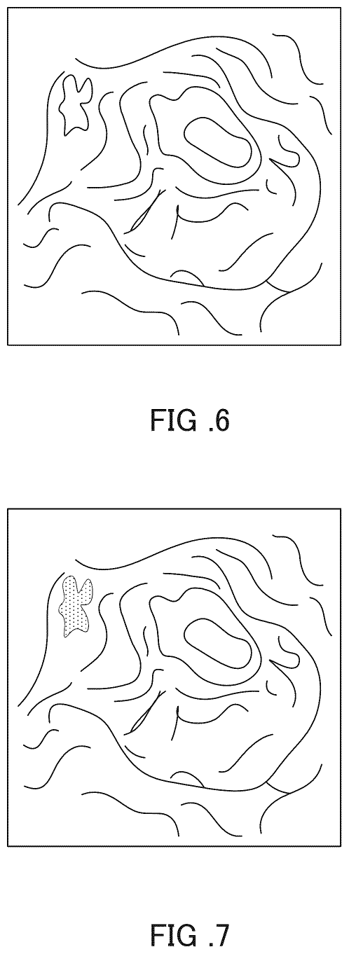

For example, the endoscopic image shown in FIG. 6 is displayed on the monitor 4 as shown in FIG. 7, with the lesion area superimposed in an enhanced manner on the endoscopic image. In the case of the detection type where the identification target is in units of rectangular frames, rather than the segmentation type where the identification is performed in units of pixels of the image, the lesion area is displayed as a rectangular frame.

The identification section 13 may output the positive probability P without performing the determination processing S25B. For example, in the segmentation type, a probability map may be created, and the positive probability may be displayed with a color nuance on the probability map, or the probability map may be superimposed on the endoscopic image.

As long as the determination result can be notified to the user, a classification type may also be used, in which the determination result is displayed as a character showing the probability that the image includes a lesion area, in an area different from the endoscopic image display area on the monitor 4. If the determination result is greater than or equal to a predetermined probability, the user may be notified with a warning sound.

### <Step S27>

The processings from step S20 are repeated until there is a user instruction. That is, the image processing apparatus 1 works continuously in real time until the endoscopy completes.

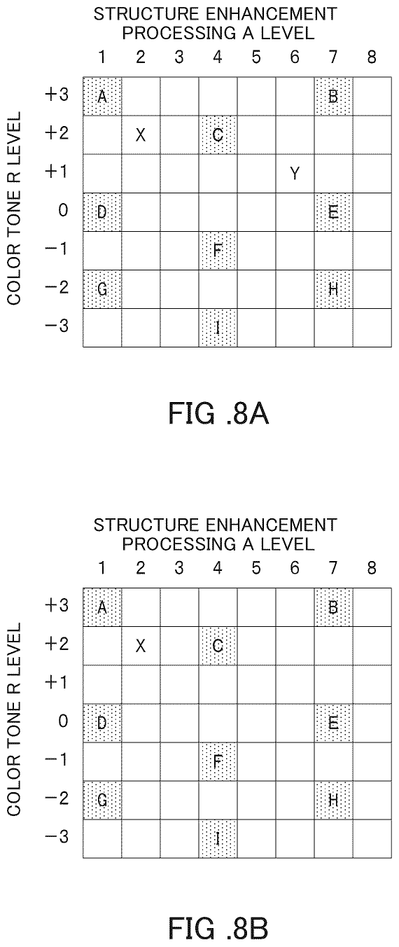

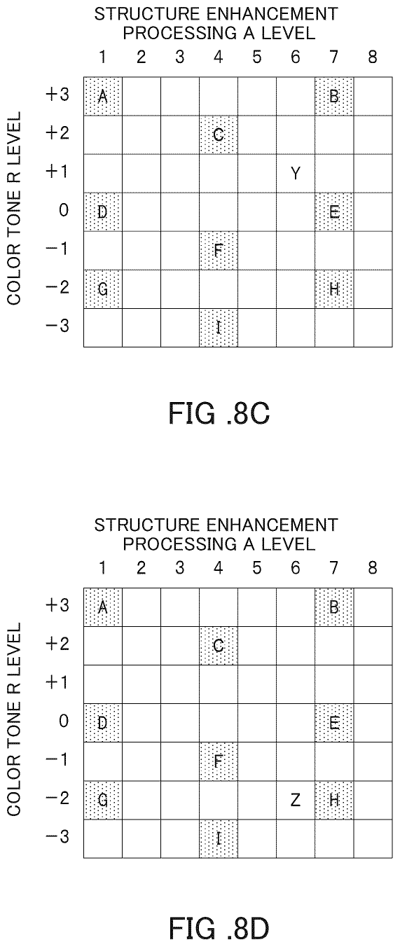

Next, specific examples of the identification processing by the image processing apparatus 1 will be described using FIGS. 8A to 8D. In FIGS. 8A to 8D, the image processing apparatus 1 stores models (first machine learning models) for nine second parameter sets from fifty-six first parameter sets that can be placed in the setting space.

For example, for images of a parameter set U observed by a physician at a medical institution U, the identification processing is performed using the models corresponding to the parameter sets A, C, and D (the second machine learning models).

In contrast, for images of a parameter set V observed with an endoscope of a physician at a medical institution V, the identification processing is performed using the models corresponding to the parameter sets B, C, and E.

For example, in the case shown in FIG. 8A, for images of a parameter set X observed with an endoscope of a model number X, the identification processing is performed using the models corresponding to the parameter sets A, C, and D (the second machine learning models).

In contrast, for images of a parameter set Y observed with an endoscope of a model number Y, the identification processing is performed using the models corresponding to the parameter sets B, C, and E.

Note that, in order to further improve the identification accuracy, a setting space may be set up for each model, organ, or the like. For example, the setting space shown in FIG. 8B corresponds to an endoscopic image of the esophagus, and if the endoscopic image has the parameter set X, the identification processing is performed using the models corresponding to the parameter sets A, C, and D.

The setting space shown in FIG. 8C corresponds to an image of the stomach taken with the white light, and if the endoscopic image has the parameter set Y, the identification processing is performed using the models corresponding to the parameter sets B, C, and E, or the models corresponding to the parameter sets B, E, and F.

The setting space shown in FIG. 8D corresponds to an image of the stomach taken with the NBI light, and if four fourth parameter sets are selected and the endoscopic image has a parameter set Z, the identification processing is performed using the models corresponding to parameter sets E, F, H, and I.

As described above, the processor 10 receives an image for identification inputted as the identification target. The processor 10 selects, from among the plurality of first machine learning models, the plurality of second machine learning models that, in the setting space, are either within a predetermined range from the image for identification, or in order of shorter distance from the image for identification. The processor 10 synthesizes the selected plurality of second machine learning models based on respective synthesis coefficients, to thereby create the synthesis model. The processor 10 uses the synthesis model to identify the image for identification. That is, the processor 10 identifies the image for identification using the selected plurality of second machine learning models.

In other words, the storage section 20 of the image processing apparatus 1 stores the plurality of first machine learning models that respectively correspond to the first images obtained in the plurality of second parameter sets selected from a plurality of maximum number of first parameter sets that can be placed in the image-parameter setting space having a plurality of parameter axes. The calculation section 12 calculates distances in the setting space between the plurality of second parameter sets and the third parameter set of the image for identification inputted as the identification target. The selection section 11 selects, from among the plurality of second parameter sets, the plurality of fourth parameter sets that are within a predetermined range or in order of shorter distance. The identification section 13 identifies the image for identification using the plurality of second machine learning models corresponding to respective second images obtained in the plurality of fourth parameter sets.

The calculation section 12 of the image processing apparatus 1 further calculates respective model synthesis coefficients of the plurality of second machine learning models corresponding to the respective second images obtained in the selected plurality of fourth parameter sets. The synthesis section 14 synthesizes the plurality of second machine learning models using the respective model synthesis coefficients, to thereby create a synthesis model. The identification section 13 uses the synthesis model to identify the image for identification.

In machine-learning (including deep learning), if there is a difference in setting conditions (parameter sets) between the training data and the data for decision making, the identification accuracy decreases.

An image parameter set is a combination of setting conditions such as photographing parameters and image processing parameters. Therefore, in order to ensure high identification accuracy in AI identification processing of images, it may be preferable to create a dedicated model using dedicated training data for each parameter set, which is a combination of a plurality of parameters.

However, the creation of many models requires the preparation of large amounts of training data and long learning times. The hardware load to store many models is also significant.

It is conceivable to learn training data of various parameter sets by a single model. However, a model that has learned training data of various parameter sets is not optimized for any of the parameter sets, generally resulting in low accuracy.

The image processing apparatus 1 ensures identification reliability even when the apparatus cannot use a model corresponding to the same parameters as those of the inputted image for identification. Since it is not necessary to create (learn) and store many models, the image processing apparatus 1 can minimize the number of machine learning models mounted and output highly accurate determination results.

<Second Embodiment>Image Processing Apparatus 1A

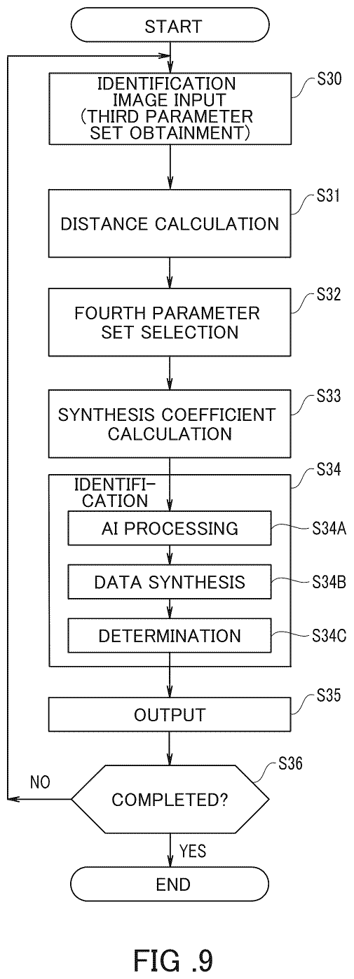

Identification processing in the image processing apparatus 1A according to the present embodiment will be described along the flowchart in FIG. 9. Since the image processing apparatus 1A is similar to the image processing apparatus 1, constituent elements with the same functions are marked with the same reference numerals, and descriptions thereof will be omitted.

In the image processing apparatus 1A, the processor 10 selects, from among the plurality of first machine learning models, the plurality of second machine learning models that, in the setting space, are within a predetermined range from the image for identification. The processor 10 uses each of the selected plurality of second machine learning models to identify the image for identification, to thereby output a plurality of positive probabilities (identification results). The processor 10 synthesizes the outputted plurality of positive probabilities (identification results) based on respective synthesis coefficients.

In other words, in the image processing apparatus 1A, the selection section 11 selects the plurality of fourth parameter sets. The calculation section 12 calculates respective positive-probability synthesis coefficients k corresponding to second images obtained in the selected plurality of fourth parameter sets. The identification section 13 outputs a plurality of positive probabilities using each of the plurality of machine learning models corresponding to the selected plurality of fourth parameter sets, and synthesizes the plurality of positive probabilities using the respective synthesis coefficients k.

Since the creation method of the machine learning model (steps S10 to S13) and steps S30 to S33 are the same as those of the image processing apparatus 1 shown in FIGS. 2 and 3, descriptions thereof will be omitted. <Step S34>Identification Processing

The image processing apparatus 1A performs identification processing (AI processing) using a plurality of models corresponding to respective images of the plurality of fourth parameter sets (S34A).

In the example in FIG. 5, the identification section 13 uses the four second models A, B, D, and E corresponding respectively to the fourth parameter sets A, B, D, and E, to perform, four times, the identification processing (AI processing) of the image for identification of the third parameter set X (S34A).

The synthesis section 14 is a positive-probability synthesis section that synthesizes positive probabilities PA, PB, PD, and PE outputted by the four second models A, B, D, and E, using respective synthesis coefficients (positive-probability synthesis coefficients) kA, kB, kD, and kE (S34B). The positive-probability synthesis coefficients are weighted according to the distance L. The calculation method for the positive-probability synthesis coefficients is substantially the same as the already described method for calculating the model synthesis coefficients, or the like.

Descriptions will be made hereinafter using an exemplary case where the positive probability P of a certain area of an image is as follows.

$$Model A ( kA = 4 / 14 ) : PA = 0.8$$

$$Model B ( kB = 4 / 14 ) : PB = 0.7$$

$$Model D ( kD = 3 / 14 ) : PD = 0.6$$

$$Model E ( kE = 3 / 14 ) : PE = 0.5$$

$$PT = PA * kA + PB * kB + PD * kD + PE * kE = 0.66$$

Because the synthesized positive probability PT is greater than or equal to a predetermined threshold (e.g., 0.5), the determination section 15 determines that the area described above corresponds to a lesion region (S34C).

Since steps S35 and S36 are the same as steps S26 and S27 of the image processing apparatus 1 shown in FIG. 3, descriptions thereof will be omitted.

As with the image processing apparatus 1, the image processing apparatus 1A ensures identification reliability even when the apparatus cannot use a model corresponding to the same parameters as those of the inputted image for identification. Since it is not necessary to create (learn) and store many models, the image processing apparatus 1A can minimize the number of machine learning models mounted and output highly accurate determination results.

### <Third Embodiment> Image Processing Apparatus 1B

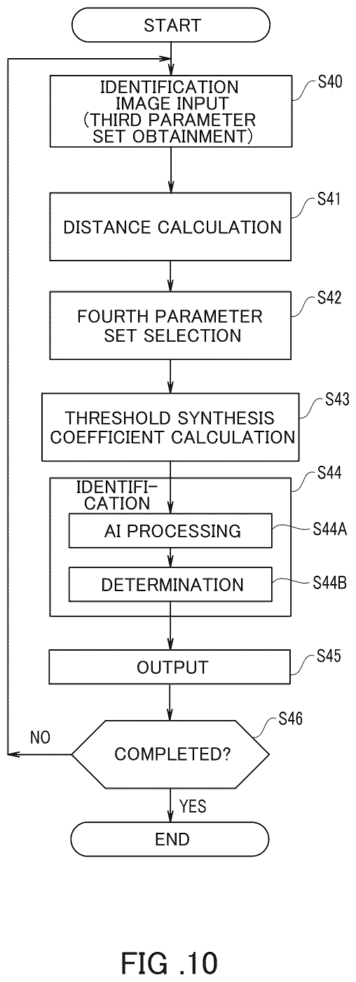

Identification processing in the image processing apparatus 1B according to the present embodiment will be described along the flowchart in FIG. 10. Since the image processing apparatus 1B is similar to the image processing apparatuses 1 and 1A, constituent elements with the same functions are marked with the same reference numerals, and descriptions thereof will be omitted.

The processor 10 of the image processing apparatus 1B stores, in the setting space including the first setting condition and the second setting condition, a plurality of first thresholds, the number of which is less than the total number of combinations of the first setting condition and the second setting condition, where the plurality of first thresholds are placed at or below a predetermined density in the setting space. The processor 10 uses one machine learning model, which is a predetermined representative model, to identify the image for identification, to thereby output a positive probability (identification result). The processor 10 selects, from among the plurality of first thresholds, a plurality of second thresholds that, in the setting space, are either within a predetermined range from the image for identification, or in order of shorter distance from the image for identification. The processor 10 determines the outputted positive probability (identification result) based on a synthesis threshold obtained by synthesizing the plurality of second thresholds.

In other words, the image processing apparatus 1B calculates, in the setting space, a threshold synthesis coefficient from the second thresholds of the plurality of fourth parameter sets according to the distance between the third parameter set and each of the selected plurality of fourth parameter sets, and uses the threshold synthesis coefficient to identify the image for identification.

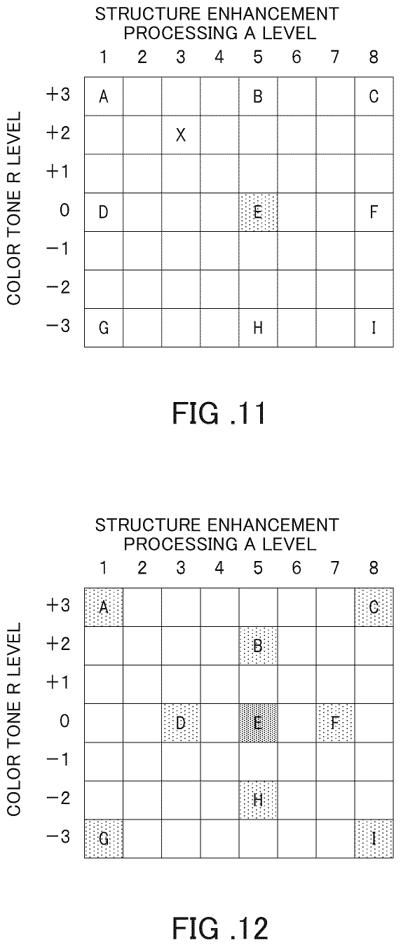

As shown in FIG. 11, the storage section 20 of the image processing apparatus 1B stores only one representative model E corresponding to an image of one preset parameter set E. The storage section 20 stores first thresholds TA to TI corresponding respectively to the selected second parameter sets A to I. The representative model E may be, for example, a preset parameter set or a frequently used parameter set. The first thresholds TA to TI are obtained in advance.

The selection section 11 of the image processing apparatus 1B selects the plurality of fourth parameter sets A, B, D, and E. The calculation section 12 calculates the threshold synthesis coefficients kA, kB, kD, and kE for the selected plurality of fourth parameter sets A, B, D, and E, respectively. The threshold synthesis coefficients are weighted according to the distance L. The method for calculating the threshold synthesis coefficients is substantially the same as the already described method for calculating the model synthesis coefficients, or the like.

The synthesis section 14 is a threshold synthesis section that creates a synthesis threshold TT by synthesizing respective thresholds (second thresholds) TA, TB, TD, and TE of the plurality of fourth parameter sets A, B, D, and E based on the respective threshold synthesis coefficients kA, kB, kD, and kE. The identification section 13 treats the image for identification using a predetermined representative model, and identifies the positive probability using the synthesis threshold TT.

Since steps S40 to S42 are the same as those of the image processing apparatus 1 shown in FIG. 3, descriptions thereof will be omitted. A second threshold Tis set for each of the fourth parameter sets selected in step S42.

As already described, the first thresholds TA to TI are respectively set in advance for the second parameter sets A to I of the image processing apparatus 1B. Of the first thresholds TA to TI, the second thresholds TA, TB, TD, and TE of the fourth parameter sets are used in the following computations.

The second thresholds TA, TB, TD, and TE for the selected fourth parameter sets A, B, D, and E will be shown as examples.

$$Parameter set A ( A 1 , R + 3 ) : threshold TA = 0.8$$

$$Parameter set B ( A 5 , R + 3 ) : threshold TB = 0.7$$

$$Parameter set D ( A 1 , R 0 ) : threshold TD = 0.6$$

$$Parameter set E ( A 5 , R 0 ) : threshold TE = 0.5$$

### <Step S43> Synthesis Threshold Calculation

The calculation section 12 calculates the synthesis threshold TT from respective distances LA, LB, LD, and LE in the setting space between the third parameter set X and the fourth parameter sets A, B, D, and E. The synthesis threshold TT is weighted so that the shorter the distance L, the greater the weight of the threshold of the fourth parameter set.

$$Synthesis threshold TT = 0.8 * ( 4 / 14 ) + 0.7 * ( 4 / 14 ) + 0.6 * ( 3 / 14 ) + 0.5 * ( 3 / 14 ) = 0.66$$

### <Step S44> Identification

The identification section 13 uses the representative model E to perform AI processing on the image for identification, and outputs a positive probability PE (S44A). For example, if the positive probability PE is 0.7, the determination section 15 determines that the area described above corresponds to the lesion region since the outputted positive probability PE is greater than or equal to the synthesis threshold TT (0.66) (S44B).

### <Step S45> Output

The processor 10 displays, on the monitor 4, the endoscopic image on which is superimposed, as a determination result, an estimated value estimated from the positive probability and the synthesis threshold on the endoscopic image.

As with the image processing apparatus 1, the image processing apparatus 1B ensures identification reliability even when the apparatus cannot use a model corresponding to the same parameters as those of the inputted image for identification. Because only one model is created, the image processing apparatus 1B can minimize the number of machine learning models mounted and output highly accurate determination results.

### MODIFICATION EXAMPLES

Since image processing apparatuses 1C to 1G of the modification examples are similar to the image processing apparatus 1, or the like, constituent elements with the same functions are marked with the same reference numerals, and descriptions thereof will be omitted.

### <First Modification Example> Image Processing Apparatus 1C

As shown in FIG. 5, in the image processing apparatus 1 or the like, the plurality of fourth parameter sets A, B, D, and E are selected in the setting space from among the second parameter sets A to I. In contrast, in the image processing apparatus 1C of the present modification example, one fourth parameter set (e.g., the parameter set B) with the shortest distance L from the third parameter set is selected. The selected one fourth parameter set B is used to perform the identification processing.

When compared with the image processing apparatus 1, the image processing apparatus 1C has lower identification reliability, but is more efficient and has a smaller hardware load.

### <Second Modification Example> Image Processing Apparatus 1D

In the image processing apparatus 1D, the method for selecting the fourth parameter set in the selection section 11 differs from that in the image processing apparatus 1 or the like.

The selection section 11 of the image processing apparatus 1D selects, from among the plurality of second parameter sets A to I, all the plurality of fourth parameter sets that fall within a predetermined range from the third parameter set of the image for identification.

For example, the selection section 11 selects, as the fourth parameter sets, the second parameter sets whose distance L from the third parameter set is less than or equal to four. In this case, the number of the fourth parameter sets selected for the image processing apparatus 1D is four, the same as the number of the fourth parameter sets for the image processing apparatus 1 or the like.

### <Third Modification Example> Image Processing Apparatus 1E

In the image processing apparatus 1, the second parameter sets are placed in the setting space in a substantially uniformly dispersed manner (FIG. 4). In contrast, as shown in FIG. 12, in the image processing apparatus 1E of the present modification example, the plurality of second parameter sets A to I are placed in the setting space such that areas with a shorter distance L from the position of the preset representative parameter set E are more densely placed than areas with a longer distance L from the position of the preset representative parameter set E.

The representative parameter set E is, for example, the most frequently used parameter set that is set by the user in advance.

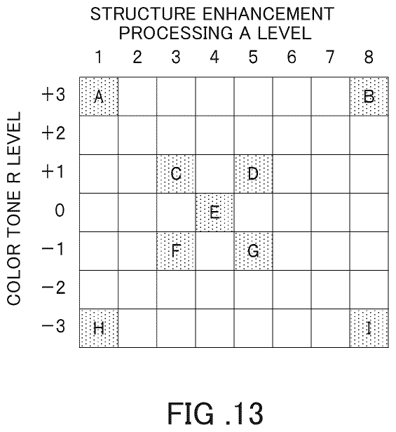

That is, for example, in the setting space, more of the second parameter sets may be placed at positions that are expected to be used more frequently than at other positions. In this case, an advantage is that high accuracy outputs can be achieved for the majority of identification targets. FIG. 13 shows an example of the placement of this case. In this case, of the parameter sets A to I, parameter sets C, D, E, F, and G are expected to be used frequently. In other words, the second parameter sets are placed in this manner if the identification target is expected to have parameters at the parameter sets C, D, E, F, and G, or at parameters in their vicinities.

The image processing apparatus 1E is more likely than the image processing apparatus 1 to be able to select a fourth parameter set at a short distance L from the third parameter set of the image for identification in the setting space. Therefore, the image processing apparatus 1E can achieve more reliable identification processing than the image processing apparatus 1.

### <Fourth Modification Example> Image Processing Apparatus 1F

The image processing apparatus 1F of the present modification example has a combination of the function of the image processing apparatus 1 and the function of the image processing apparatus 1B.

The processor 10 of the image processing apparatus 1F stores, in the setting space including the first setting condition and the second setting condition, one or more first machine learning models and a plurality of first thresholds, the numbers of which are less than the total number of combinations of the first setting condition and the second setting condition. The one or more first machine learning models and the plurality of first thresholds are placed at or below a predetermined density in the setting space. The processor 10 receives an image for identification inputted as the identification target. The processor 10 selects, from among the one or more first machine learning models and the plurality of first thresholds, one or more second machine learning models and a plurality of second threshold that, in the setting space, are either within a predetermined range from the image for identification, or in order of shorter distance from the image for identification. The processor 10 identifies the image for identification using the one or more second machine learning models and the plurality of second thresholds.

For example, the storage section 20 of the image processing apparatus 1F stores machine learning models A, B, D, and E and thresholds TA, kB, kD, and kE corresponding to respective second images obtained in the plurality of fourth parameter sets A, B, D, and E.

As with the image processing apparatus 1, in the image processing apparatus 1F, AI processing is performed on the image for identification using a synthesis model obtained by synthesizing the machine learning models A, B, D, and E, and the positive probability is outputted.

As with the image processing apparatus 1B, for the processing for determining the positive probability, a synthesis threshold obtained by synthesizing the thresholds TA, kB, kD, and kE is used.

### <Fifth Modification Example> Image Processing Apparatus 1G

The image processing apparatus 1G of the present modification example has a combination of the function of image processing apparatus 1A and the function of the image processing apparatus 1B.

The storage section 20 of the image processing apparatus 1G stores the machine learning models A, B, D, and E and the threshold synthesis coefficients kA, kB, kD, and kE corresponding to the respective second images obtained in the plurality of fourth parameter sets A, B, D, and E.

As with the image processing apparatus 1A, in the image processing apparatus 1G, the processor 10 uses the machine learning models A, B, D, and E to identify the image for identification, to thereby calculate respective positive probabilities and calculate a synthesized positive probability.

As with the image processing apparatus 1B, for the processing for determining the positive probability, the machine learning models A, B, D, and E are synthesized based on the respective threshold synthesis coefficients kA, kB, kD, and kE, and the synthesized positive probability is determined using the synthesis threshold TT.

In the image processing apparatuses 1 and 1A to 1G described above, the image for identification is a medical image, and the identification section 13 identifies a lesion region.

When used in the embodiments and modification examples described above, the machine learning models can also be applied to automatic switching model techniques.

For example, the image for identification changes in the process in which the endoscope is inserted from the mouth, advanced through the pharynx, the upper esophagus, the lower esophagus, and the stomach, and then returned to the mouth. This may be because the color tone of the mucosa in each region is different, or because the observation light is changed from the white light mode to the special light mode when the stomach is observed.

For example, the preferable model for the image for identification differs between an image of the lower esophagus and an image of the stomach, but with the present disclosure, an appropriate model or a synthesis model can be applied even if the observation region changes.

That is, the images for identification (third images) are endoscopic images obtained continuously, and the identification section 13 performs the identification processing by automatically selecting a machine learning model according to changes in the plurality of parameters of the endoscopic images.

The image for identification may be a medical image such as a pathology image that has been obtained in advance and stored in the external storage apparatus 30, for example. That is, the image processing apparatus need not be directly connected to the video processor 3, but may be indirectly connected to the video processor 3 via the external storage apparatus 30 or the like.

The image may be an endoscopic image, and the identification section 13 may identify the position of a distal end portion of the endoscope, which is the position in the body where the image for identification was photographed.

For example, an insertion navigation system is disclosed that forms a three-dimensional image of a bronchus in the lungs from three-dimensional image data of an examinee acquired by a CT apparatus or the like, finds a path to a target point along the lumen on the three-dimensional image, and further generates and displays a virtual endoscopic image of the lumen based on the three-dimensional image data. The image processing apparatus selects a virtual endoscopic image that matches the endoscopic image of the bronchoscope inserted into the lung, to allow identifying the position where the image for identification was photographed (the distal end position of the endoscope in the body).

Note that the endoscope in the embodiments may be a flexible endoscope having a flexible insertion portion or a rigid endoscope having a rigid insertion portion. The endoscope according to the embodiments may be used for medical or industrial purposes.

In an image processing method according to an embodiment, a processor stores, in a setting space including a first setting condition and a second setting condition, at least one of: one or more first machine learning models, the number of which is less than a total number of combinations of the first setting condition and the second setting condition; or a plurality of first thresholds, the number of which is less than the total number of combinations of the first setting condition and the second setting condition. The at least one of the one or more first machine learning models or the plurality of first thresholds is placed at or below a predetermined density in the setting space. The processor receives an image for identification inputted as an identification target. The processor selects, from among the at least one of the one or more first machine learning models or the plurality of first thresholds, at least one of: one or more second machine learning models that, in the setting space, are either within a predetermined range from the image for identification, or in order of shorter distance from the image for identification; or a plurality of second thresholds that, in the setting space, are either within the predetermined range from the image for identification, or in order of shorter distance from the image for identification. The processor identifies the image for identification using the at least one of the one or more second machine learning models or the plurality of second thresholds.

An image processing program according to an embodiment is configured such that a processor of the image processing apparatus stores, in a setting space including a first setting condition and a second setting condition, at least one of: one or more first machine learning models, the number of which is less than a total number of combinations of the first setting condition and the second setting condition; or a plurality of first thresholds, the number of which is less than the total number of combinations of the first setting condition and the second setting condition. The at least one of the one or more first machine learning models or the plurality of first thresholds is configured to be placed at or below a predetermined density in the setting space. The processor is configured to receive an image for identification inputted as an identification target. The processor is configured to select, from among the at least one of the one or more first machine learning models or the plurality of first thresholds, at least one of: one or more second machine learning models that, in the setting space, are either within a predetermined range from the image for identification, or in order of shorter distance from the image for identification; or a plurality of second thresholds that, in the setting space, are either within the predetermined range from the image for identification, or in order of shorter distance from the image for identification. The processor is configured to identify the image for identification using the at least one of the one or more second machine learning models or the plurality of second thresholds.

The program may be stored and distributed on a non-transitory computer-readable storage medium such as a magnetic disk (floppy (registered trademark) disk, hard disk, etc.), an optical disk (CD-ROM, DVD, etc.), a magneto-optical disk (MO), or a semiconductor memory. Any storage format may be used for the storage medium, as long as the medium can store the program and is computer-readable.

Based on instructions of a program installed on the computer from the storage medium, an OS (operating system), middleware such as database management software, network software, or the like, operating on the computer may execute some of the processings to achieve the embodiments described above.

The storage medium is not limited to a medium independent of the computer, but also includes a storage medium in which a program transmitted over a LAN, the Internet, or the like is downloaded and stored or temporarily stored. The storage medium is not limited to a single medium, but the storage medium in the present disclosure also includes cases where each of the processings described above is executed from a plurality of media, and the medium may take any configuration.

Note that the computer in each embodiment executes each processing in each of the embodiments described above, based on a program stored in a storage medium, and may take any configuration such as an apparatus consisting of a single personal computer or the like, and a system in which a plurality of apparatuses are connected in a network.

The term “computer” is not limited to personal computers, but also includes a computation processing apparatus, a microcomputer, and the like included in information processing instrument, and collectively refers to any instrument or apparatus that can realize the functions of the present disclosure by means of a program.

The present disclosure is not limited to the embodiments and modification examples described above, but various changes, alterations, and the like are possible within the scope without departing from the gist of the present disclosure.

What is claimed is:

1. An image processing apparatus comprising one or more processors, wherein the one or more processors store,
in a setting space including a first setting condition and a second setting condition, at least one of:
one or more first machine learning models, the number of which is less than a total number of combinations of the first setting condition and the second setting condition; or
a plurality of first thresholds,
the number of which is less than the total number of combinations of the first setting condition and the second setting condition, the at least one of the one or more first machine learning models or the plurality of first thresholds being placed at or below a predetermined density in the setting space,
the one or more processors receive an image for identification inputted as an identification target,
the one or more processors select, from among the at least one of the one or more first machine learning models or the plurality of first thresholds, at least one of:
one or more second machine learning models that, in the setting space, are either within a predetermined range from the image for identification, or in order of shorter distance from the image for identification; or a plurality of second thresholds that, in the setting space, are either within the predetermined range from the image for identification, or in order of shorter distance from the image for identification, and
the one or more processors identify the image for identification using the at least one of the one or more second machine learning models or the plurality of second thresholds.

2. The image processing apparatus according to claim 1, wherein
the one or more processors select a plurality of second machine learning models,
the one or more processors synthesize the plurality of second machine learning models based on respective synthesis coefficients, to thereby create a synthesis model, and
the one or more processors use the synthesis model to identify the image for identification.

3. The image processing apparatus according to claim 1, wherein
the one or more processors select a plurality of second machine learning models,
the one or more processors use each of the plurality of second machine learning models to identify the image for identification, to thereby output a plurality of identification results, and
the one or more processors synthesize the outputted plurality of identification results based on respective synthesis coefficients.

4. The image processing apparatus according to claim 1, wherein
the one or more processors identify the image for identification using a predetermined machine learning model, to thereby output an identification result,
the one or more processors select a plurality of second thresholds that, in the setting space, are either within a predetermined range from the image for identification, or in order of shorter distance from the image for identification, and
the one or more processors determine the outputted identification result based on a synthesis threshold obtained by synthesizing the plurality of second thresholds.

5. The image processing apparatus according to claim 2, wherein the one or more processors calculates the synthesis coefficients according to a distance, in the setting space, between the image for identification and the at least one of the one or more second machine learning models or the plurality of second thresholds.

6. The image processing apparatus according to claim 2, wherein the one or more processors calculate, based on parameter information possessed by an endoscope or an endoscope processor, a distance between the image for identification and the one or more second machine learning models in the setting space.

7. The image processing apparatus according to claim 1, wherein the machine learning models are placed uniformly in terms of at least one of the first setting condition or the second setting condition.

8. The image processing apparatus according to claim 1, wherein
the one or more processors store a plurality of machine learning models, and
a placement density in the setting space of at least one of the plurality of machine learning models or the plurality of thresholds is less than or equal to 50%.

9. The image processing apparatus according to claim 1, wherein a placement density in the setting space of at least one of the plurality of first machine learning models or the plurality of first thresholds is higher the closer to a position of a preset representative parameter set.

10. The image processing apparatus according to claim 1, wherein by learning only an image of an arbitrary setting condition, a first machine learning model optimal for the arbitrary setting condition is created.

11. The image processing apparatus according to claim 1, wherein the one or more processors select one second machine learning model having the shortest distance from the image for identification in the setting space.

12. The image processing apparatus according to claim 1, wherein
the image for identification is a medical image, and
the one or more processors identify a lesion region.

13. The image processing apparatus according to claim 1, wherein
the image for identification is an endoscopic image, and
the image for identification is outputted from an endoscope processor.

14. The image processing apparatus according to claim 1, wherein
the image for identification comprises endoscopic images obtained continuously, and
the one or more processors perform identification processing by automatically selecting the at least one of the one or more second machine learning models or the plurality of second thresholds, according to changes in the first setting condition and the second setting condition of the endoscopic images.

15. The image processing apparatus according to claim 2, wherein
the machine learning model comprises a plurality of layers, and
the synthesis coefficients differ for each of the plurality of layers.

16. An image processing method comprising:
storing, in a setting space including a first setting condition and a second setting condition, at least one of: one or more first machine learning models, the number of which is less than a total number of combinations of the first setting condition and the second setting condition; or a plurality of first thresholds, the number of which is less than the total number of combinations of the first setting condition and the second setting condition, the at least one of the one or more first machine learning models or the plurality of first thresholds being placed at or below a predetermined density in the setting space;
receiving an image for identification inputted as an identification target;
selecting, from among the at least one of the one or more first machine learning models or the plurality of first thresholds, at least one of: one or more second machine learning models that, in the setting space, are either within a predetermined range from the image for identification, or in order of shorter distance from the image for identification; or a plurality of second thresholds that, in the setting space, are either within the predetermined range from the image for identification, or in order of shorter distance from the image for identification; and
identifying the image for identification using the at least one of the one or more second machine learning models or the plurality of second thresholds.

17. A non-transitory computer-readable storage medium storing a program for an image processing apparatus, wherein
a one or more processors of the image processing apparatus stores, in a setting space including a first setting condition and a second setting condition, at least one of: one or more first machine learning models, the number of which is less than a total number of combinations of the first setting condition and the second setting condition; or a plurality of first thresholds, the number of which is less than the total number of combinations of the first setting condition and the second setting condition, the at least one of the one or more first machine learning models or the plurality of first thresholds being placed at or below a predetermined density in the setting space, and
the program is configured such that:
the one or more processors receive an image for identification inputted as an identification target;
the one or more processors select, from among the at least one of the one or more first machine learning models or the plurality of first thresholds, at least one of: one or more second machine learning models that, in the setting space, are either within a predetermined range from the image for identification, or in order of shorter distance from the image for identification; or a plurality of second thresholds that, in the setting space, are either within the predetermined range from the image for identification, or in order of shorter distance from the image for identification; and
the one or more processors identify the image for identification using the at least one of the one or more second machine learning models or the plurality of second thresholds.
